(19) United States
(12) Patent Application Publication
(10) Pub. No.: US 2005/0079546 A1
(43) Pub. Date: Apr. 14, 2005

(54) Serum albumin scaffold-based proteins and uses thereof

(76) Inventors: Dasa Lipovsek, Cambridge, MA (US); Lin Sun, West Roxbury, MA (US); Alexander V. Kovtun, Acton, MA (US)

Correspondence Address: FISH & NEAVE IP GROUP;ROPES & GRAY LLP, ONE INTERNATIONAL PLACE, BOSTON, MA, 02110-2624, US

(21) Appl. No.: 10/838,311

(22) Filed: May 3, 2004

Related U.S. Application Data
(60) Provisional application No. 60/466,957, filed on May 1, 2003.

(51) Int. Cl. G01N 33/53; G01N 33/543; C07K 14/765

(52) U.S. Cl. 435/7.1; 436/518; 530/363

(57) ABSTRACT

Disclosed herein are libraries of candidate binding proteins derived from a serum albumin scaffold, from which useful binding proteins may be selected that are specific for known and unknown targets. Also disclosed herein are methods for selection of serum albumin scaffold-based candidate binding proteins which possess such desired binding characteristics.

### RELATED APPLICATION

This application claims the benefit of U.S. Provisional Patent Application 60/466,957, filed on May 1, 2003, the entire content of which is hereby incorporated by reference.

### BACKGROUND OF THE INVENTION

Serum albumin is a protein found in plasma that plays a role in numerous physiological activities. For example, serum albumin is involved in the regulation of the osmotic pressure in blood. Serum albumin can also act as a carrier for endogenous and exogenous ligands such as hormones or drugs (Bertucci et al., Curr. Med. Chem. 9:1463, 2002).

The stability of human serum albumin (HSA) in the bloodstream has been used to extend the half life of protein drugs by attaching them covalently (Osborn et al., J. Pharmacol. Exp. Ther. 303:540, 2002) or non-covalently (Dennis et al., J. Biol. Chem. 277:35035, 2002) to HSA. HSA is also known to bind to many small-molecule drugs, and to release them into the bloodstream gradually, improving their pharmacokinetic properties.

### SUMMARY OF THE INVENTION

In general, this invention relates to serum albumin scaffold-based proteins and uses thereof. The invention involves the use of serum albumin (for example, HSA) as a protein scaffold. Although serum albumin is a very abundant and well-studied protein, its structure bears little resemblance to the antibody fold on which most protein scaffolds are based. Despite this lack of resemblance, we have discovered that serum albumin, particularly various domains and sub-domains of HSA, has a structure that is quite amenable to mutation or randomization for the generation of serum albumin scaffold-based protein libraries. As this scaffold is based on a naturally occurring protein, it is expected to be stable in the bloodstream and well tolerated by the immune system and therefore effective not only as a diagnostic or research tool but as a potential therapeutic protein as well.

Accordingly, one aspect of the present invention features a library of serum albumin scaffold-based candidate binding proteins, the proteins having sequences derived from a serum albumin, preferably the HSA protein sequence of FIG. 1 (SEQ ID NO: 1), wherein each of the candidate binding proteins has one or more mutated amino acids within at least one of the serum albumin domains or sub-domains.

In preferred embodiments, at least one candidate binding protein in the library has a mutation in sub-domain IB, sub-domain IIB, sub-domain IIIB, sub-domain IA, sub-domain IIA, sub-domain IIIA, domain I, domain II, or domain III of HSA, or any combination thereof. At least one candidate binding protein may, in preferred embodiments, have a mutation in full-length HSA, or may be fill-length or less than full-length and have mutations in more than one HSA sub-domain, for example, mutations in two or more amino acids or six or more amino acids, but preferably in no more than 50% of the total amino acids. In yet other preferred embodiments, the at least one candidate binding protein has a mutated HSA sub-domain fused to the remaining wild-type HSA protein or has a mutated HSA sub-domain fused to at least one additional HSA sub-domain.

In preferred libraries, at least one candidate binding protein has a binding affinity for a compound or target that is at least ten fold higher than the binding affinity of wild-type HSA for the compound. A preferred compound or target is tumor necrosis factor alpha (TNF-α). Another preferred compound is vascular endothelial growth facto (VEGF). Other preferred targets include, but are not limited to, VEGF receptor (e.g., KDR), interleukin 1 (IL-1), and IL-1 receptor.

In other preferred libraries, at least one candidate binding protein is part of a fusion protein, for example, fused to a complement protein or a toxin protein.

Also provided are peptidomimetics based on a serum albumin scaffold-based protein of the invention.

The candidate binding proteins of the library may be immobilized on a solid support, such as a chip or bead. The candidate binding proteins of the library may also be part of an array immobilized on a solid support.

The invention also features a library of nucleic acid-protein fusion molecules, the proteins of the molecules being derived from the HSA sequence of FIG. 1 (SEQ ID NO: 1), wherein each of the proteins has one or more mutated amino acids within at least one of the serum albumin domains or sub-domains, the proteins being attached to the nucleic acids of the fusion molecules by means of a covalent bond.

In preferred embodiments, the nucleic acid includes DNA and/or RNA (for example, mRNA); and/or the nucleic acid of the fusion molecule encodes the protein to which it is bound. The nucleic acid-protein fusion molecules of the library may be immobilized on a solid support, such as a chip or bead; and may be part of an array immobilized on a solid support.

Another aspect of the invention employs a library of the invention to obtain one or more proteins that bind to a compound of interest, such as for example TNF-α or VEGF. In one method, binding proteins are obtained by: (a) contacting a compound with a library of serum albumin scaffold-based candidate binding proteins under conditions that allow binding to form a compound-protein complex, the proteins being derived from the wild-type HSA protein sequence of FIG. 1 (SEQ ID NO: 1), wherein each of the candidate binding proteins has one or more mutated amino acids within at least one of the serum albumin domains or sub-domains; and (b) obtaining, from the complex, a protein that binds to the compound.

In preferred embodiments, the method further involves mutating at least one sub-domain of the protein obtained in step (b) and repeating steps (a) and (b) using the further mutated protein. Preferably, the compound is a protein, such as for example TNF-α or VEGF. In preferred methods, the candidate binding proteins may be covalently bound to a nucleic acid (for example, DNA or RNA) and/or the nucleic acid may encode the protein to which it is bound. The candidate binding proteins of the library may be immobilized on a solid support, such as a chip or bead, and the candidate binding proteins of the library may be part of an array immobilized on a solid support.

Another aspect of the invention employs a library of the invention to obtain one or more proteins that has a biological activity of interest, such as for example enzymatic activity, enzyme substrate activity, regulating signaling (e.g., gene expression) in a cell, or being an agonistic or antagonistic ligand for a particular receptor, channel, or other membrane proteins. In one method, biologically active proteins are obtained by screening a library of serum albumin scaffold-based candidate binding proteins, wherein each of the candidate binding proteins is derived from the wild-type HSA protein sequence of FIG. 1 (SEQ ID NO: 1) and has one or more mutated amino acids within at least one of the serum albumin domains or sub-domains, for a serum albumin scaffold-based protein having a desired biological activity measured by an assay, such as for example a receptor- or membrane protein-binding assay or an assay measuring receptor-mediated signaling in a cell. In preferred embodiments, the method further involves mutating at least one sub-domain of the protein obtained by a screening assay using the further mutated protein.

A further aspect of the invention features a composition comprising a pharmaceutically acceptable carrier and a serum albumin scaffold-based protein of the invention that binds to a compound of interest, such as for example TNF-α or VEGF, or has a biological activity of interest. The invention further provides methods of administering such compositions, for example to a patient in need thereof.

### BRIEF DESCRIPTION OF THE DRAWINGS

FIG. 1 shows the amino acid sequence of full-length human serum albumin from Protein Data Bank: 1E7A.

FIG. 2A shows the amino acid sequence of each of the human serum albumin sub-domains.

FIG. 2B shows the relevant characteristics of each sub-domain.

FIG. 3 shows the crystal structure of full-length human serum albumin (PDB: 1E7A).

FIG. 4 shows the crystal structures of human serum albumin sub-domains IB, IIB, and IIIB.

FIG. 5 shows the crystal structures of human serum albumin sub-domains IIA, IIIA, IB, IB, and IIB, each of which is suitable for randomization.

FIG. 6 shows, as underlined sequences, the randomizable sequence elements of each human serum albumin sub-domain.

FIG. 7 shows the structure of each of the human serum albumin domains and sub-domains used for the serum albumin scaffold-based protein constructs described in the examples provided herein. The domains and sub-domains are shown in the context of the full-length HSA.

FIG. 8A shows a schematic of the final cDNA construct used for serum albumin scaffold-based RNA-protein fusion formation as described in the examples provided herein.

FIG. 8B shows the primers and predicted fragment sizes for the PCR products generated for each of the cDNA constructs. FIG. 8C shows a photograph of a gel containing the PCR products after amplification of HSA domains and sub-domains.

FIG. 9 shows a photograph of an overnight exposure of a NuPAGE 4-12% Bis-Tris gel containing each of the eight ³⁵S labeled serum albumin scaffold-based RNA protein fusions after in vitro translation.

FIG. 10A shows a photograph of a gel containing the DNA products after 15 cycles of PCR amplification of the flow-through from a binding experiment using the control PR 1 Hf02 library and TNF-α or VEGF as targets. FIG. 10B shows a photograph of a gel containing the DNA products after 15 cycles of PCR amplification of the flow-through from the simulated binding experiment using the eight serum albumin scaffold-based RNA-protein fusions.

FIG. 11A shows a photograph of a gel containing the DNA products after 20 cycles of PCR amplification of the flow-through from a binding experiment using the control PR1 Hf02 library and TNF-α or VEGF. FIG. 11B shows a photograph of a gel containing the DNA products after 20 cycles of PCR amplification of the flow-through from the simulated binding experiment using the eight serum albumin scaffold-based RNA-protein fusions.

### DETAILED DESCRIPTION OF THE INVENTION

1. Overview

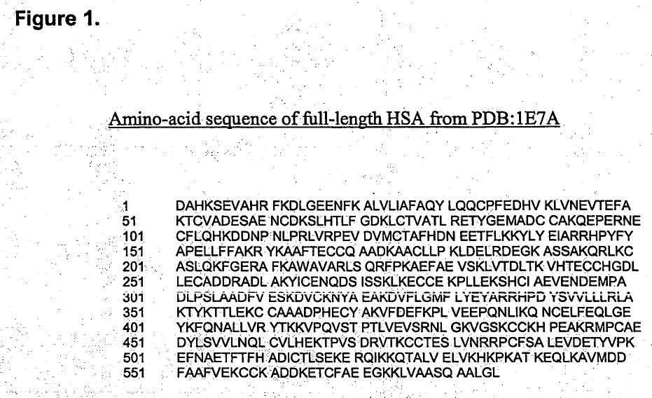

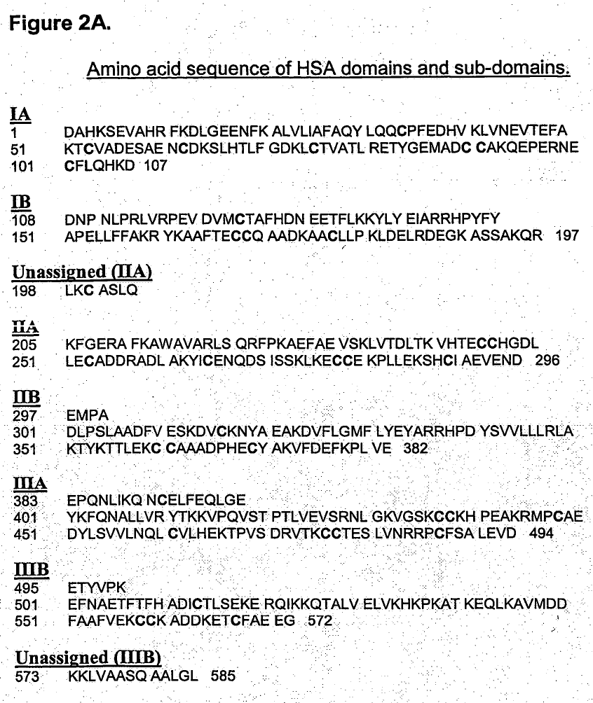

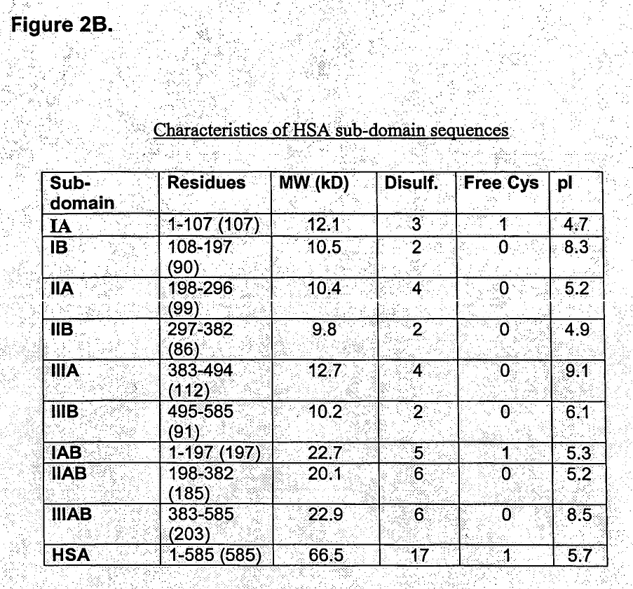

Human serum albumin (HSA) is the most abundant protein in human plasma. It is retained in the plasma due to its relatively high molecular weight and the stability of its tertiary structure. HSA contains 585 amino acid residues (FIG. 1), a large percentage of which are ionic, making the protein highly soluble as well. The crystal structure of HSA has been determined (e.g., Bhattacharya et al., J. Biol. Chem. 275:38731, 2000) and shows three homologous domains (I, II, III), each of which is made up of two sub-domains (IA, IB, IIA, IIB, IIIA, IIIB; FIG. 2A; boundaries of domains and sub-domains assigned by CATH (Orengo et al., Structure 5:1093, 1997)). Each of the sub-domains contains pairs of cysteine residues that are connected by disulfide bonds. These disulfide bonds provide structural constraints that contribute to the overall stability of the tertiary structure of HSA. Accordingly, the present invention provides serum albumin scaffold-based proteins derived from a serum albumin, preferably an HSA, and having at least one mutation in at least one domain or sub-domains of the serum albumin, libraries of the serum albumin scaffold-based proteins, methods of using such libraries to obtain a serum albumin scaffold-based protein that binds to a compound of interest or has a desired biological activity, and compositions comprising a pharmaceutically acceptable carrier and a serum albumin scaffold-based proteins and uses thereof.

2. Definitions

By “binding” in the context of serum albumin scaffold-based proteins is meant a covalent or non-covalent interaction between a serum albumin scaffold-based protein and a desired compound of interest, or binding partner (e.g., a protein). Such an interaction can be a low affinity or a high affinity interaction between a serum albumin scaffold-based protein and a binding partner. A high affinity interaction is preferred.

The term “biological activity” refers to a structural, regulatory, or biochemical function of a naturally occurring, recombinant or synthetic molecule. Biological activity may also include a binding activity or a particular protein-protein interaction/association.

By “candidate binding protein” in the context of this invention is meant a serum albumin scaffold-based protein that has the potential to bind to a particular compound. Non-limiting examples of desirable compounds include proteins (e.g., enzymes, antigens, antibodies, receptors), nucleic acids (e.g., DNA, RNA), and any drugs or small molecule compounds.

By a “fusion protein” in the context of this invention is meant a serum albumin scaffold-based protein joined to a second, different or heterologous protein. “Fusion proteins” are distinguished from “nucleic acid-protein fusions” in that a “fusion protein” is composed entirely of amino acids, while a “nucleic acid-protein fusion” includes a stretch of nucleic acids joined to a stretch of amino acids (the protein component); in preferred embodiments, at least a portion of the stretch of nucleic acid encodes at least a portion of the stretch of amino acids. It is also contemplated that a fusion protein may also be the protein component of a nucleic acid-protein fusion molecule.

By “library of serum albumin scaffold-based proteins,” as used herein, is meant a collection of serum albumin scaffold-based candidate binding proteins or fragments thereof. A collection includes at least 10² serum albumin scaffold-based proteins, preferably at least 10³ or 10⁴ serum albumin scaffold-based proteins, more preferably, 1×10⁶, 1×10⁸, or 1×10¹⁰ serum albumin scaffold-based proteins, and most preferably at least 1×10¹² serum albumin scaffold-based proteins.

By “mutated” or “randomized” is meant including one or more amino acid alterations relative to a template sequence. Alterations can include any substitution of an amino acid within a protein sequence to another related or unrelated amino acid. Alterations can also include the insertion of any amino acid or protein sequence that does not decrease the overall structural stability of serum albumin. Preferably the insertions will not be proximal to the N- or C-terminus of a protein, e.g., serum albumin. More preferably, the insertion will be in the loops that are close to the disulfide bonds or the exposed surface of the protein. By “mutating” or “randomizing” is meant the process of introducing, into a sequence, one or more such amino acid alterations. Mutating or randomizing may be accomplished through intentional, blind, or spontaneous sequence variation, generally of a nucleic acid coding sequence, and may occur by any technique, for example, PCR, error-prone PCR, or chemical DNA synthesis. By a “corresponding, non-mutated protein” is meant a protein that is identical in sequence, except for the introduced amino acid mutations. Although any number of amino acid residues can be mutated, it is preferable that less than 100 amino acids are mutated in each scaffold derivative, more preferably less than 50 amino acids, and most preferably less than 25 amino acids. The number of amino acid residues that can be mutated within each sub-domain can also vary. Preferably, less than 50 amino acids are mutated in any sub-domain, more preferably less than 25, and most preferably less than 15 amino acids are mutated or randomized in any sub-domain.

By “randomizing” is also meant the process of introducing one or more amino acid alterations which may be randomly selected from any one of the twenty known natural amino acids. The resulting sequence is considered “randomized.” By “randomizing a sub-domain or a domain” is meant that the amino acid sequence of a naturally occurring sub-domain or domain is replaced by a “mutated” or “randomized” amino acid sequence, and optionally the amino acid at each position of which is randomly selected from any one of the twenty known natural amino acids.

By a “nucleic acid” is meant any two or more covalently bonded nucleotides or nucleotide analogs or derivatives. As used herein, this term includes, without limitation, DNA, RNA, and PNA. By “DNA” is meant a sequence of two or more covalently bonded, naturally occurring or modified deoxyribonucleotides. By “RNA” is meant a sequence of two or more covalently bonded, naturally occurring or modified ribonucleotides. One example of a modified RNA included within this term is phosphorothioate RNA. By “PNA” is meant an analog of DNA in which the pentose sugar unit backbone is replaced with a pseudopeptide backbone made from repeating N-(2-aminoethyl)-glycine units linked by peptide bonds.

By a “protein” is meant any sequence of two or more amino acids joined by a peptide bond(s), regardless of length, post-translation modification, or function. “Protein,” “peptide,” and “polypeptide” are used interchangeably herein.

By “pharmaceutically acceptable” is meant capable of administration to an animal (for example, a mammal) without significant adverse medical consequences. One exemplary pharmaceutically acceptable protein drug is insulin. Other pharmaceutically acceptable drugs and their formulations are known to one skilled in the art and are described, for example, in Remington's Pharmaceutical Sciences, (20th ed., ed. A. R. Gennaro, Lippincott Williams & Wilkins, Philadelphia, Pa., 2000), incorporated herein by reference.

By “scaffold” is meant a protein having a framework with specific and favorable properties, such as binding. When designing proteins from the scaffold, e.g., designing a scaffold-based protein, amino acid residues that are important for the framework's favorable properties (e.g., structure) are retained, while other residues may be varied. Preferably, such a scaffold-based protein has less than or equal to a 1% variation in amino acid residues between protein scaffold derivatives having different properties, more preferably less than 10% variation, more preferably less than 20% variation, and most preferably less than 50% variation between such derivatives. Preferably, the residues that confer the same overall three-dimensional fold to all the variant domains will remain constant, regardless of their properties.

By “selecting” is meant substantially partitioning a molecule with desired properties from other molecules in a population. As used herein, a “selecting” step provides at least a 2-fold, preferably, at least a 10-fold, more preferably, at least a 50-fold, and, most preferably, at least a 100-fold enrichment of a desired molecule relative to undesired molecules in a population following the selection step. A selection step may be repeated any number of times, and different types of selection steps may be combined in a given approach.

By “serum albumin” is meant any protein found in the plasma of an animal that bears substantial similarity to the amino acid sequence of HSA (GenBank Accession Number AAN17825) or SEQ ID NO: 1 and functions in osmotic pressure regulation or acts as a serum carrier for endogenous and exogenous ligands such as hormones or drug. Non-limiting examples of serum albumin proteins from various animal species include monkey (GenBank Accession Number A47391), cow (GenBank Accession Number AAN17824), dog (GenBank Accession Number BAC10663), mouse (GenBank Accession Number CAD29888), and rat (GenBank Accession Number NP_{—}599153).

By “substantially identical” or “substantially similar” is meant a polypeptide or nucleic acid exhibiting at least 50%, but preferably 75%, more preferably 90%, most preferably 95%, or even 99% identity to a reference amino acid or nucleic acid sequence. For polypeptides, the length of comparison sequences will generally be at least 20 amino acids, preferably at least 30 amino acids, more preferably at least 40 amino acids, and most preferably 50 amino acids.

By “domain of human serum albumin” is meant any of the three domains identified based on the crystal structure of HSA (e.g., I, II, III; Protein Databank Number IE7A, Bhattacharya et al., J. Biol. Chem. 275:38731, 2000). By “sub-domain of human serum albumin” is meant any of the six smaller domains found within the three domains (e.g., IA, IB, IIA, 1113, IIIA, 11113). The amino acid sequence of each of the domains and sub-domains of human serum albumin is found in FIG. 2A. By “domain” or “sub-domain of serum albumin” is meant a domain or sub-domain in the scrum albumin protein that corresponds to the domain or sub-domain in the HSA protein.

By “solid support” is meant, without limitation, any column (or column material), bead, test tube, micro-titer dish, solid particle (for example, agarose or sepharose), microchip (for example, silicon, silicon-glass, or gold chip), fiber optic-based arrays (e.g., BeadArray™ by Illumina, Inc.), or membrane (for example, an inorganic membrane, nitrocellulose, or the membrane of a liposome or vesicle) to which a molecule of the invention may be bound, either directly or indirectly, or in which a molecule of the invention may be embedded (for example, through a receptor or channel).

By an “array” is meant is meant a fixed pattern of immobilized objects on a solid surface or membrane. The array preferably includes at least 10², more preferably at least 10³, and most preferably at least 10⁴ different members, and these members are preferably arrayed on a 125×80 mm, and more preferably on a 10×10 mm, surface.

Other features and advantages of the present invention will be apparent from the following detailed description thereof, and from the claims.

3. Scaffold Proteins and Libraries

The present invention provides a serum albumin protein scaffold from which libraries of mutated or randomized proteins can be developed, facilitating the generation of a variety of candidate proteins of a biological activity of interest, preferably candidate binding proteins for known and unknown targets such as proteins (e.g., enzymes and receptors) and small molecules. Desired or useful binders can be selected in vitro or in vivo. The scaffolds described herein are based on the serum albumin protein, preferably HSA, and bear little structural resemblance to the antibody fold on which most protein scaffolds are based. There are several major advantages of the HSA based-scaffold over previously known antibody and non-antibody based scaffolds. For example, HSA has a very stable tertiary structure, is soluble and stable in the bloodstream, and is well tolerated by the immune system, all of which are desirable characteristics for the development of proteins of pharmaceutical interest. HSA contains three distinct domains and six sub-domains, each of which can be modified, mutated or randomized for purposes of the present invention, allowing for a great deal of diversity in candidate protein or binder libraries or for constructing protein arrays for the in vitro screening assays, such as those described herein.

The libraries of the present invention include collections of serum albumin scaffold-based candidate binding proteins. A collection includes at least 10² serum albumin scaffold-based proteins, preferably at least 1×10³ or 1×10⁴ serum albumin scaffold-based proteins, more preferably, at least 1×10⁶, 1×10⁸, or 1×10¹⁰ serum albumin scaffold-based proteins, and most preferably at least 1×10¹² serum albumin scaffold-based proteins. Preferably, the serum albumin scaffold-based candidate binding proteins are derived from wild-type serum albumin and mutated or randomized, for example, as described below. Such serum albumin scaffold-based proteins are amenable to any selection method, for example, any method of in vitro selection, such as selection using RNA-protein fusions (RNA display) as described below or other display methodologies, such as phage display (See, e.g., Brian Kay et al., Phage Display of Peptides and Proteins: A Laboratory Manual, Academic Press, 1^{st} ed., January 1996), ribosome display (See, e.g., Amstutz et al., Curr. Opin. Biotechnol. (2001) 12, 400-405; Plückthun et al., Curr. Opin. Biotechnol. (2000) 12, 400-405.), or yeast display (e.g., Yeast Display Expression Kit made by Invitrogen). Thus, the present invention provides libraries of mutated or randomized serum albumin scaffold-based proteins, wherein any amino acid, in any sub-domain or domain, or combination of sub-domains or domains, may be mutated or randomized. The libraries of the present invention can comprise any one or a combination of randomized or mutated serum albumin scaffold-based proteins or fragments thereof, serum albumin scaffold-based protein fusions, nucleic acid-serum albumin scaffold-based protein fusions, or multimers of the scaffold proteins as described herein.

4. Human Serum Albumin (HSA)

The serum albumin scaffold-based protein libraries of the present preferably include a collection of serum albumin scaffold-based candidate proteins, preferably candidate binding proteins, derived from HSA. While the detailed description presented herein specifically refers to HSA, it will be clear to one skilled in the art that the detailed description can also apply to serum albumin from any other animal species. Non-limiting examples of additional animal species from which the serum albumin can be derived include monkey, chimpanzee, cow, pig, dog, cat, rabbit, mouse, rat, and frog.

The crystal structure of HSA has been determined and shows three homologous domains (I, II, III), each of which is made up of two sub-domains (IA, IIA, IIIA, IB, IIB, IIIB; FIG. 2A). HSA has cysteine residues, which are connected in pairs within each sub-domain by 17 disulfide bonds. The one free cysteine residue (Cys34) is found in sub-domain IA. Mao et al. (Protein Expr. Purif. 20: 492, 2000) have shown that all three domains can be expressed, purified, and refolded in E. coli, but that only domains I and III are stable by themselves.

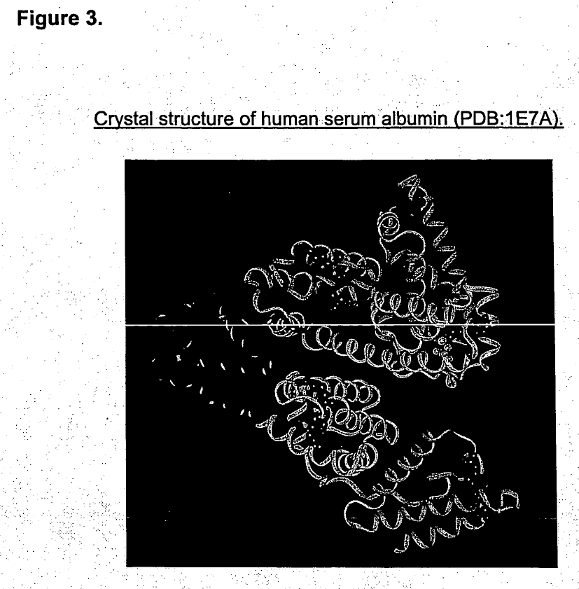

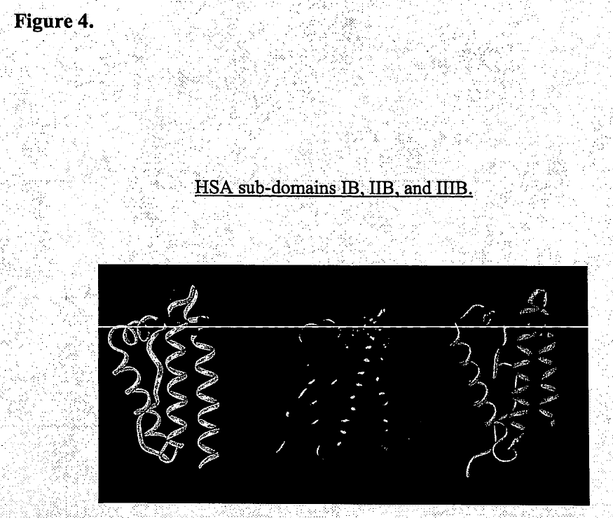

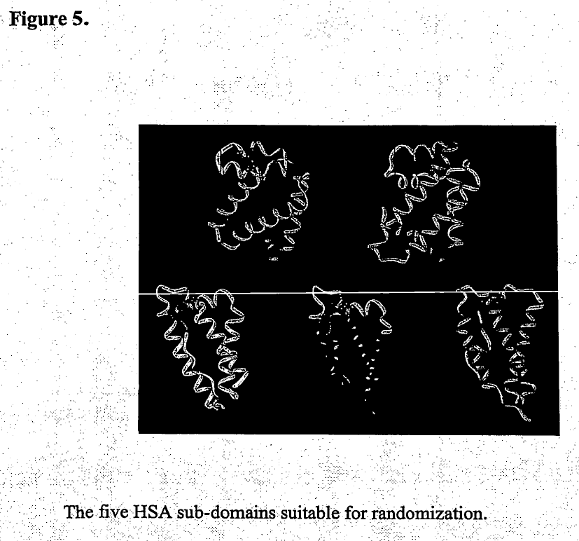

The structural stability provided by the disulfide bonds in each of the sub-domains makes them amenable to sequence mutation or randomization. Sub-domains IB, IIB, and IIIB are the simplest folded elements of HSA. Each of these sub-domains is defined by three alpha helices, which run anti-parallel to each other, and by two disulfide bonds. Disulfide bonds in HSA sub-domains B are found close to the surface of full-length HSA (FIG. 3). The disulfide bonds occur in pairs centered on two cysteines adjacent in the primary sequence (e.g., Cys 168 and Cys 169 in domain IB). Each of the two cysteines forms a disulfide bond with a cysteine on a different adjacent alpha helix. The resulting structure includes a loop constrained by two disulfides, and short alpha helices or loops that run between a disulfide and a major secondary structural element (FIG. 4). It is likely that this constrained loop structure allows for changes in the amino acid sequence between the helices without a significant structural effect on the rest of the molecule, thus making it an ideal location for generating mutations or for performing sequence randomizations.

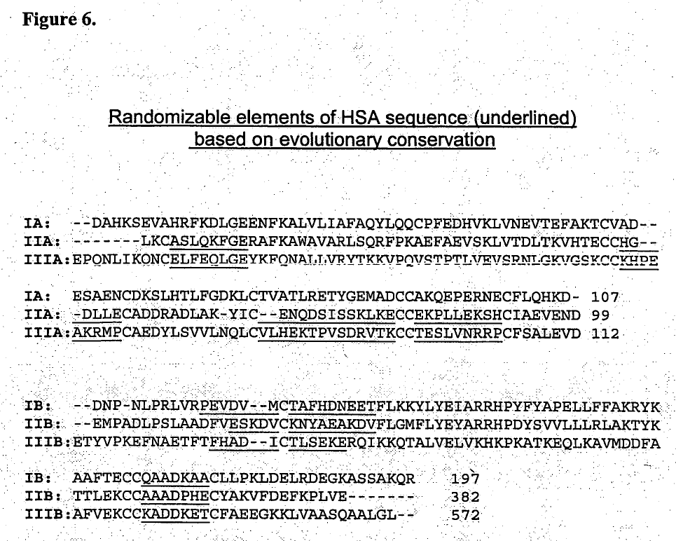

In order to generate a library of HSA scaffold-based candidate binding proteins, sequence variation may be introduced into any amino acid of the wild-type serum albumin sequence using standard mutagenesis techniques including, for example, PCR-based mutagenesis by Taq polymerase (Tindall and Kunkel, Biochemistry 27:6008, 1998), fragment recombination, oligonucleotide synthesis, or a combination thereof. Similarly, an increase in the structural diversity of libraries, for example, can be achieved by varying the length as well as the sequence of the HSA sub-domains or domains. The most preferred region for generating sequence changes is in the loops between the two cysteines (e.g., amino acids 170-176 in sub-domain IB, amino acids 362-368 in sub-domain IIB, and amino acids 560-566 in sub-domain IIIB) as these domains are the most tolerant, structurally, of changes to the amino acid sequence or sequence length. Additional preferred regions for sequence alterations include the short stretches of residues that are not part of the anti-parallel alpha helices as listed in Table 1 and in FIG. 6.

HSA sub-domains IIA and IIIA contain two pairs of cysteines each, similar to those found in the B sub-domains. Every pair of cysteines makes a disulfide bond to a cysteine residue close to the N- or C-terminus of the domain, consequently forming only two loops suitable for mutation. HSA sub-domains IIA and IIIA, however, do contain disulfide-restrained loop structures, as well as short stretches of residues that are not part of the anti-parallel alpha helices, each of which would be optimal regions for mutation. For example, amino acid residues 247-252 of sub-domain IIA or amino acid residues 439-447 of sub-domain IIIA are preferred regions for sequence mutation or randomization. Amino acid residues 201-208 of sub-domain IIA and 393-400 of sub-domain IIIA are also preferred. See Table 2 and FIG. 6 for additional regions of HSA sub-domains IIA and IIIA suitable for randomization.

TABLE 1
Summary of amino acid regions of HSA B sub-domains
particularly suitable for randomization.

| HSA IB | HSA IIB | HSA IIIB |
|---|---|---|
| AA: 170-176 | AA: 362-368 | AA: 560-566 |
| Disulfide-restrained | Disulfide-restrained loop | Disulfide-restrained loop |
| loop | | |
| AA: 125-133 | AA: 317-325 | AA: 515-520 |
| 2-turn alpha helix | 2-turn alpha helix | Loop/turn |
| AA: 118-123 | AA: 311-315 | AA: 509-513 |
| Loop | Loop/turn | Loop/turn |

AA = amino acids

TABLE 2
Summary of amino acid regions of HSA A sub-domains
particularly suitable for randomization.

| HSA IIA | HSA IIIA |
|---|---|
| AA: 247-252 | AA: 439-447 |
| Disulfide-restrained loop | Disulfide-restrained loop |
| AA: 201-208 | AA: 393-400 |
| 2-turn α helix | Loop/turn |
| AA: 280-288 | AA: 478-486 |
| Disulfide-restrained loop | Disulfide-restrained loop |
| AA: 268-277 | AA: 462-475 |
| 2-turn α helix + turn | 2-turn α helix + 2-turn α helix |

AA = amino acids

As noted above, any domain or sub-domain of HSA can be mutated at any amino acid position. Each of the sub-domains of HSA can be altered individually or in combination with other sub-domains for use as a serum albumin scaffold-based candidate protein in the methods of the present invention. One sub-domain, two sub-domains, three sub-domains, four sub-domains, five sub-domains, or all six sub-domains can be altered simultaneously to produce an optimal scaffold protein for the desired methods. The preferred sub-domains for sequence alteration or randomization include sub-domain IB and sub-domains IIIA and IIIB. One preferred combination of sub-domains for generating sequence alterations or mutations is sub-domains IIIA and IIIB (i.e., domain III). Any part of domains I and II are also preferred regions for generating mutations.

Once the mutated or altered sub-domain is generated, it may be fused, or cloned, back into the gene encoding the remainder of the full-length, wild-type HSA protein using standard recombinant DNA technology (see, Maniatis et al. (1989) Molecular Cloning: A Laboratory Manual, Cold Spring Harbor, N.Y. and Ausubel et al., Current Protocols in Molecular Biology, Greene Publishing Associates, New York, V. 1 &2 1996, incorporated by reference herein). The resulting protein is a serum albumin scaffold-based protein with the natural size and overall shape of HSA, but with a new target-binding site in the case of a candidate binding protein (or a new biologically active site in the case of a candidate protein of a particular biological activity) in one or more of the domains. The mutated sub-domain or sub-domains can also be expressed as a single sub-domain, combination of sub-domains, or fused with a portion or fragment of the HSA protein resulting in a HSA scaffold-based protein. In addition, sequences can be added or deleted from any of the sub-domains or from the full-length HSA protein in order to enhance the solubility, stability, expression of the protein, or to improve binding. Once the recombinant DNA encoding the HSA scaffold-based protein, or fragment thereof, is generated, it may be expressed in a eukaryotic expression system such as yeast or mammalian cell culture using standard protein expression techniques (see Maniatis et al., supra, and Ausubel et al., supra). Bacterial cultures can also be used for the expression of the serum albumin scaffold-based proteins, or fragments thereof. Alternatively, the serum albumin scaffold-based proteins may be expressed by various display methodologies, such as for example phage display, E. coli display, yeast display, or ribosome display.

5. Serum Albumin Scaffold-Based Fusion Proteins, Nucleic Acid-Protein Fusions, and Other Variants

The libraries of serum albumin scaffold-based candidate binding proteins described herein can also include a collection of serum albumin scaffold-based proteins or fragments thereof fused to other protein domains. For example, a fusion between a serum albumin scaffold-based protein and a complement protein, such as C1q, can be used to target cells. A fusion between a serum albumin scaffold-based protein and a toxin can be used to specifically destroy cells that carry a particular antigen; preferably the antigen being recognized or subject to binding by the serum albumin scaffold-based protein. Any of these fusions can be generated by standard techniques, for example, by expression of the fusion protein from a recombinant fusion gene constructed using publicly available gene sequences.

The present invention also provides fusion molecules that include an HSA scaffold-based protein attached to a nucleic acid molecule. Preferably, attachment occurs via a covalent bond. In particularly preferred embodiments, the nucleic acid attached to serum albumin scaffold-based protein encodes the serum albumin scaffold-based protein to which it is attached. The nucleic acid molecule may be a DNA molecule or an RNA molecule, preferably an mRNA molecule. In one example, the serum albumin scaffold-based protein is attached to the 3′ end of an mRNA by means of a covalent bond (e.g., an amide bond). Upon translation, a serum albumin scaffold-based protein is generated that has, at one end, the entire mRNA sequence encoding the serum albumin scaffold-based protein. It will be appreciated by one of ordinary skill in the art that the serum albumin scaffold-based protein of the fusion molecules can be randomized, as described above. Exemplary methods for generating nucleic acid-serum albumin scaffold-based peptide fusions molecules can be found in Szostak et al., U.S. Pat. Nos. 6,258,558 and 6,261,804; and Lohse et al., U.S. Pat. No. 6,416,950.

Other variants of the serum albumin scaffold-based proteins include peptidomimetics based on a serum albumin scaffold-based protein of the invention. Peptidomimetics are compounds in which at least a portion of a subject serum albumin scaffold-based protein of the invention is modified, and the three dimensional structure of the peptidomimetic remains substantially the same as that of the subject protein. Peptidomimetics may be analogues of a subject protein of the invention that are, themselves, polypeptides containing one or more substitutions or other modifications within the subject protein sequence. Alternatively, at least a portion of the subject protein sequence may be replaced with a nonpeptide structure, such that the three-dimensional structure is substantially retained. In other words, one, two or three amino acid residues within the subject polypeptide sequence may be replaced by a non-peptide structure. In addition, other peptide portions of the subject protein may, but need not, be replaced with a non-peptide structure. Peptidomimetics (both peptide and non-peptidyl analogues) may have improved properties (e.g., decreased proteolysis, increased retention or increased bioavailability). Peptidomimetics generally have improved oral availability, which makes them especially suited to treatment of disorders in a human or animal. It should be noted that peptidomimetics may or may not have similar two-dimensional chemical structures, but share common three-dimensional structural features and geometry. Each peptidomimetic may further have one or more unique additional binding elements.

6. Scaffold Multimers

The libraries of serum albumin scaffold-based candidate binding proteins described herein can also include a collection of two or more serum albumin scaffold-based proteins generated as dimers or multimers of serum albumin scaffold-based proteins or fragments thereof as a means to increase the valency and thus the avidity of target binding. Such multimers may be generated through covalent binding. In particular examples, covalently bonded multimers may be generated by constructing fusion genes that encode the multimer or, alternatively, by engineering codons for cysteine residues into monomer sequences or relying on naturally-occurring cysteine residues, and allowing disulfide bond formation to occur between the expression products. Non-covalently bonded multimers may also be generated by a variety of techniques. These include the introduction, into monomer sequences, of codons corresponding to positively and/or negatively charged residues and allowing interactions between these residues in the expression products (and therefore between the monomers) to occur. This approach may be simplified by taking advantage of charged residues naturally present in a monomer subunit, for example, the charged residues of HSA. Another means for generating non-covalently bonded serum albumin scaffold-based proteins is to introduce, into the monomer gene (for example, at the amino- or carboxy-termini), the coding sequences for proteins or protein domains known to interact with the monomer protein. Such proteins or protein domains include coil-coil motifs, leucine zipper motifs, and any of the numerous protein subunits (or fragments thereof) known to direct formation of dimers or higher order multimers. The nucleic acid-serum albumin scaffold-based scaffold proteins can also be multimerized or assembled to become multimers according to the methods disclosed in the PCT Patent Application WO03/012046.

7. Identification of Candidate Proteins or Target Binders

The unique serum albumin scaffold-based protein libraries of the invention are amenable to in vivo or in vitro selection and may be used in any technique for identification of target protein binders or target candidate protein to generate improved binding proteins or target proteins with enhanced biological activity. Any of a variety of peptide display systems known in the art (e.g., phage, yeast, ribosome, and bacterial) may be used with the serum albumin scaffold-based proteins of the present invention.

In one example, a library of serum albumin scaffold-based proteins is used to screen for specific proteins that can interact with a target compound (e.g., a protein such as for example TNF-α or VEGF). The target of binding may be immobilized on a solid support, such as a column resin or micro-titer plate well, and the target contacted with a library of candidate serum albumin scaffold-based binding proteins. Such a library may consist of randomized serum albumin scaffold-based proteins, serum albumin scaffold-based fusion proteins, or nucleic acid-serum albumin scaffold-based protein fusion molecules as described above. The library is incubated with the immobilized target, the support is washed to remove non-specific binders, and the tightest binders are eluted under stringent conditions. Binders can be subjected to PCR to recover the sequence information if nucleic acid-serum albumin scaffold-based peptide fusion molecules are used or proteins can be purified and subjected to peptide sequencing or analysis (e.g., mass spectrometry) to recover the sequence information. Binders can also be further randomized to create a new library for use in repeated selection rounds. Methods of in vitro selection include column chromatography and RNA-protein fusion selection. Methods of RNA-protein fusion selection are provided in detail in Szostak et al. (WO 98/31700; U.S. Pat. Nos. 6,258,558 and 6,261,804), incorporated herein by reference.

The invention also employs fragments, e.g., one or more domains or sub-domains of serum albumin scaffold-based proteins for screening. In preferred embodiments, a library of any of the mutated or randomized serum albumin domains or sub-domains may be used in screening for a candidate binder protein. The selected domain or sub-domain may then be fused back into the remainder of the full-length, wild-type serum albumin protein using known technologies such as standard recombinant DNA technology.

If desired, the serum albumin scaffold-based proteins may include a tag to facilitate isolation and purification of selected serum albumin scaffold-based molecules. Alternatively, the tag may be attached to the target molecule or substrate. A wide variety of tags are available commercially and in the art (see, for example, Ausubel et al., supra). To provide but a few examples, tags of the present invention include biotin tags, FLAG tags, and polyhistidine tags.

The targets for candidate binding proteins used can be any protein or compound including, but not limited to, enzymes, substrates, receptors, ligands, hormones, antigens, antibodies, peptides, or drug compounds. One preferred target protein is tumor necrosis factor alpha (TNF-α). Additional preferred targets include interleukin-1, interleukin-1 receptor, vascular endothelial growth factor (VEGF), and the VEGF receptor KDR.

A library of serum albumin scaffold-based proteins may also be screening for candidate proteins having a desired biological activity. For example, a candidate protein may bind to a membrane protein, e.g., a receptor, as an agonist or antagonist, and the activity of the candidate protein may be determined by assaying for the receptor-mediated signaling in a cell. In another embodiment, a candidate protein may have enzymatic activity, e.g., kinase or phosphatase activity, and phosphorylation assays may be used to determine the candidate protein's activity. In other embodiments, a candidate protein may be a substrate for an enzyme (e.g., a kinase), and assays (e.g., kinase assays) specific to the enzyme may be employed to determine whether the candidate protein has the desired substrate activity. In yet another embodiment, a candidate protein may regulate target gene expression in a cell, and assays (e.g., reporter-gene assays) may be employed to determine whether the candidate protein activates or inhibits target gene expression.

8. Uses

Libraries of serum albumin scaffold-based candidate binding proteins of the present invention may be screened for candidate binding proteins with optimal binding affinities to virtually any compound and are therefore useful for diagnostic, therapeutic, and research purposes. For example, in vitro selection can be used to identify scaffold molecules that display preferential binding to particular proteins (e.g., receptors or enzymes). Such preferential binders can be used as candidate drug therapies or as diagnostic tools. Multiple rounds of selection can also be used to select for mutations that enhance binding of an HSA scaffold-based protein to a particular target. Such enhanced binders may also be useful for therapeutic or diagnostic purposes. The serum albumin scaffold-based proteins of the present invention and methods disclosed herein are also useful for research purposes. For example, such proteins are useful in the study of protein-protein interactions, specific protein signaling pathways, or for the identification or particular domains required for protein binding.

The use of HSA as the basis for the generation of candidate binding proteins of the present invention also enables the adaptation of identified optimal binders for therapeutic uses. HSA is soluble and is not likely to elicit an immune response; therefore, serum albumin scaffold-based proteins of the present invention, e.g., identified as optimal binders for a particular target, may be administered as a pharmaceutically acceptable composition for therapeutic purposes.

9. Pharmaceutical Compositions

In certain embodiments, serum albumin scaffold-based proteins of the present invention are formulated with a pharmaceutically acceptable carrier. For example, a serum albumin scaffold-based protein can be administered alone or as a component of a pharmaceutical formulation (therapeutic composition). The subject proteins may be formulated for administration in any convenient way for use in human or veterinary medicine.

In certain embodiments, the therapeutic method of the invention includes administering the composition topically, systemically, or locally as an implant or device. When administered, the therapeutic composition for use in this invention is, of course, in a pyrogen-free, physiologically acceptable form. Further, the composition may desirably be encapsulated or injected in a viscous form for delivery to the various sites, e.g., sites of tissue damage. Topical administration may be suitable for wound healing and tissue repair. Therapeutically useful agents other than the subject serum albumin scaffold-based proteins which may also optionally be included in the composition as described above, may alternatively or additionally, be administered simultaneously or sequentially with the subject proteins in the methods of the invention.

In certain embodiments, methods of the invention can be administered for orally, e.g., in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) or as mouth washes and the like, each containing a predetermined amount of an agent as an active ingredient. An agent may also be administered as a bolus, electuary or paste.

In solid dosage forms for oral administration (capsules, tablets, pills, dragees, powders, granules, and the like), one or more therapeutic compounds of the present invention may be mixed with one or more pharmaceutically acceptable carriers, such as sodium citrate or dicalcium phosphate, or any of the following: (1) fillers or extenders, such as starches, lactose, sucrose, glucose, mannitol, or silicic acid; (2) binders, such as, for example, carboxymethylcellulose, alginates, gelatin, polyvinyl pyrrolidone, sucrose, or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

Certain compositions disclosed herein may be administered topically, either to skin or to mucosal membranes. The topical formulations may further include one or more of the wide variety of agents known to be effective as skin or stratum corneum penetration enhancers. Examples of these are 2-pyrrolidone, N-methyl-2-pyrrolidone, dimethylacetamide, dimethylformamide, propylene glycol, methyl or isopropyl alcohol, dimethyl sulfoxide, and azone. Additional agents may further be included to make the formulation cosmetically acceptable. Examples of these are fats, waxes, oils, dyes, fragrances, preservatives, stabilizers, and surface active agents. Keratolytic agents such as those known in the art may also be included. Examples are salicylic acid and sulfur.

Dosage forms for the topical or transdermal administration include powders, sprays, ointments, pastes, creams, lotions, gels, solutions, patches, and inhalants. The active compound may be mixed under sterile conditions with a pharmaceutically acceptable carrier, and with any preservatives, buffers, or propellants which may be required. The ointments, pastes, creams and gels may contain, in addition to a subject serum albumin scaffold-based protein of the invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a subject compound, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

In certain embodiments, pharmaceutical compositions suitable for parenteral administration may comprise one or more serum albumin scaffold-based polypeptides in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

The compositions of the invention may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

It is understood that the dosage regimen will be determined by the attending physician considering various factors which modify the action of the subject proteins of the invention. The various factors include, but are not limited to, the patient's age, sex, and diet, the severity of any infection, time of administration, and other clinical factors. The addition of other known components to the final composition, may also effect the dosage. Progress can be monitored by periodic assessment of the patient's condition.

Incorporation by Reference

All publications including patents mentioned herein are hereby incorporated by reference in their entirety as if each individual publication or patent was specifically and individually indicated to be incorporated by reference.

While specific embodiments of the subject matter have been discussed, the above specification is illustrative and not restrictive. Many variations will become apparent to those skilled in the art upon review of this specification and the claims below. The full scope of the invention should be determined by reference to the claims, along with their full scope of equivalents, and the specification, along with such variations.

### EXAMPLES

The following examples are provided for the purpose of illustrating the invention and should not be construed as limiting.

### Example 1

### Serum Albumin Scaffold-Based Protein Fusion Libraries

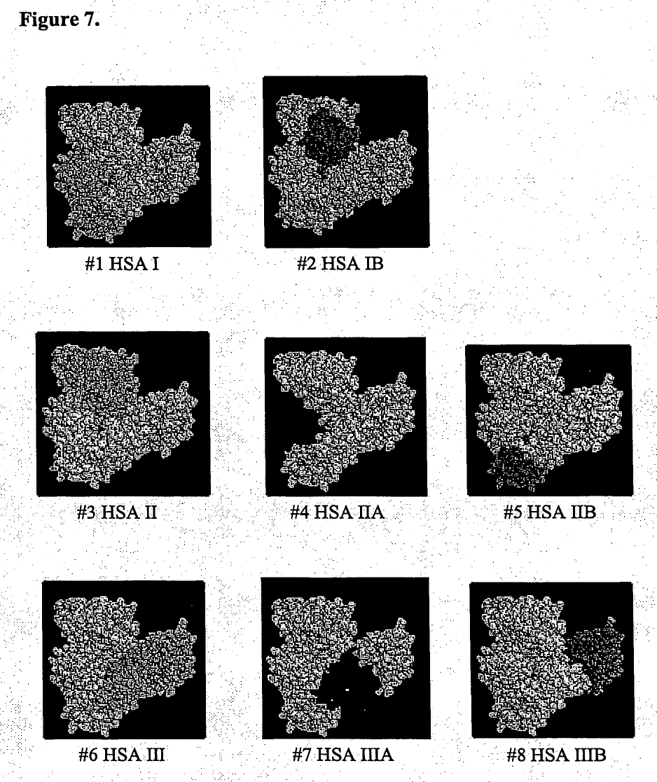

Eight different serum albumin scaffold-based protein constructs were generated using the methods and techniques of the RNA-protein fusion system (see U.S. Pat. Nos. 6,258,558 and 6,261,804, incorporated herein by reference). FIG. 7 depicts each of the eight domains and sub-domains used.

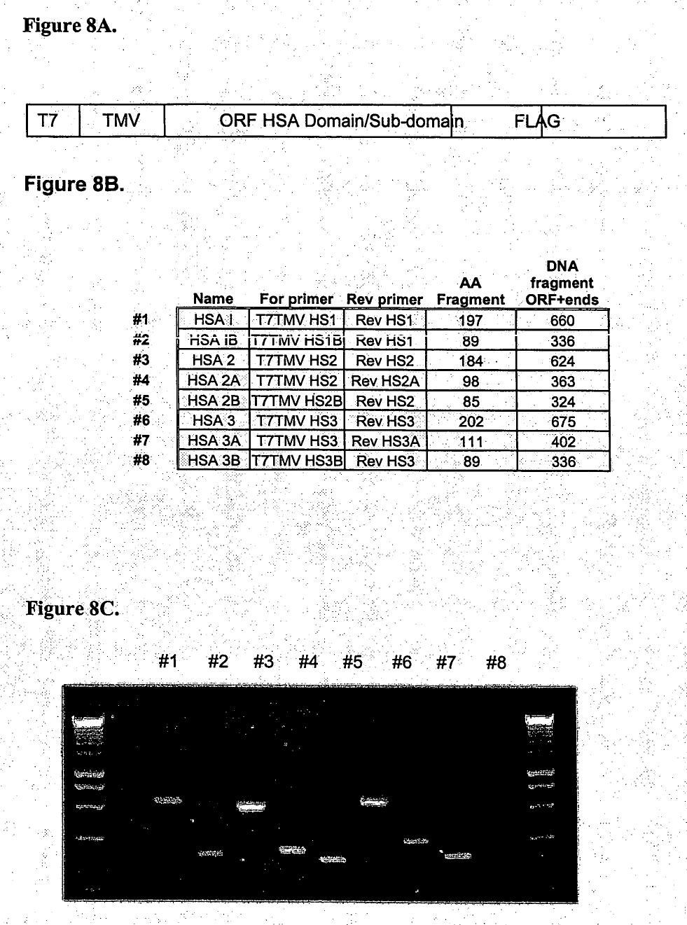

The initial step in generating the serum albumin scaffold-based RNA-protein fusions involved construction of cDNA clones for each of the eight constructs. A cDNA clone corresponding to the nucleotide sequence of HSA (GenBank Accession Number AF542069) was obtained from Invitrogen. Each of the eight constructs was created by polymerase chain reaction (PCR) using primers designed based on the sequence of the HSA cDNA clone. FIG. 8B shows the primers used and the predicted fragment length for each of the constructs. FIG. 8C shows the results of the PCR reactions for each of the HSA constructs generated. A final PCR reaction was used to make the constructs appropriate for RNA-protein fusion cDNA construct formation. A schematic of the final serum albumin scaffold-based RNA-protein fusion construct is shown in FIG. 8A.

The RNA-protein fusion cDNA constructs generated above were transcribed in vitro using a MEGA Shortscript kit from Ambion (catalog number 1354). Using each of the eight serum albumin scaffold-based fusion constructs generated above, an in vitro transcription reaction was set up using the MEGA Shortscript kit (Ambion catalog number 1354). 160 μl of PCR product was mixed with 40 μl of 10× reaction buffer (supplied with kit), 40 μl of 75 mM ATP, 40 μl of 75 mM CTP, 40 μl of 75 mM GTP, 40 μl of 75 mM UTP and 40 μl of enzyme according to the MEGA Shortscript protocol. The reaction was incubated at 37° C. for three hours, 20 μl DNAse I was added, and the reaction was incubated for an additional 15 minutes. The reaction was then subjected to phenol-chloroform extraction. The aqueous phase of the extraction was recovered as the transcription mix and loaded onto an NAP-25 column for fractionation. After allowing the flow-through to drip through, the column was washed two times with 0.8 mL dH20 and once with 1.1 mL dH20. 1.0 mL dH20 was then added and the flow-through was collected. Most of the RNA was present in this fraction (4-10 nmol). An additional 0.5 mL dH20 was added to the column and collected to retrieve any remaining RNA. The RNA concentration was measured by A₂₆₀ on a spectrophotometer. The RNA concentration for each of the eight samples is shown in Table 3, below.

TABLE 3

| | | RNA Conc. | Total |
|---|---|---|---|
| | Name | (pmol/μL) | RNA (nmoles) |
| #1 | HSA I | 6.2 | 6.2 |
| #2 | HSA IB | 14.1 | 14.1 |
| #3 | HSA II | 8.0 | 8.0 |
| #4 | HSA IIA | 12.7 | 12.7 |
| #5 | HSA IIB | 12.2 | 12.2 |
| #6 | HSA III | 7.0 | 7.0 |
| #7 | HSA IIIA | 10.5 | 10.5 |
| #8 | HSA IIIB | 9.9 | 9.9 |

The fraction containing the greatest amount of RNA for each construct was then used in a chemical ligation reaction in order to ligate the template RNA to the peptide acceptor molecule as described in U.S. Pat. Nos. 6,258,558 and 6,261,804. Briefly, 2 mmol of RNA was incubated on ice with 3 μL PEG6 linker (1 mM) and 10× chemical ligation buffer in a total volume of 400 μL. The reaction was allowed to anneal in a PCR machine at 85° C. for 30 seconds and then moved to 4° C. at the rate of 0.3° C./second. The annealed ligation mix was then transferred to a borosilicate glass vial (Kimble Glass Inc., catalog number 6094D 12) in a total volume of 400 μL/vial. The mix was then irradiated with a Handheld Multiwavelength UV lamp (uvp.com, catalog number UVGL-25) on “long wave” for 15 minutes at room temperature. After chemical ligation, each of the ligation mixes was in vitro translated using rabbit reticulocyte lysates from Ambion (catalog number PH1200, 1 mL). The reaction conditions generally consisted of 600 pmol (120 μL) of the ligated RNA mix obtained above, 75 μL of the master mix supplied with the kit which contains all amino acids except methionine, 15 μL of ³⁵S labeled methionine (15 μM), 17 μL of 100 mM Glutathione (oxidized form; Sigma, catalog number G 6654)/100 mM Glutathione (reduced form; Sigma, catalog number G 6013), 10/1 mix, 30 μL of protein disulfide isomerase (Sigma, catalog number P 3818) at 1 unit/μL, and 1000 μL rabbit reticulocyte lysate in a total volume of 1500 μL. The reaction mixture was incubated at 30° C. for one hour. 500 μL of 2 M KCl and 100 μL of 1 M MgCl2 were added and the mixture was incubated at 4° C. for 40 minutes. 235 μL of 0.5 M EDTA was added to stop the reaction and 10 μL was removed for scintillation counting to determine the “input” amounts.

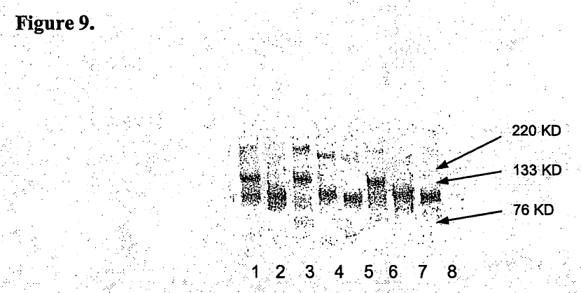

The ³⁵S labeled products were then counted in a scintillation counter and equivalent counts of each sample were separated on a 4-12% NuPAGE Bis-Tris gel and run at 50 mAmps for one hour. An overnight exposure of the gel showing each of the ³⁵S-labeled translation products is shown in FIG. 9.

Translation products were then recovered from the translation mixture using an Oligo dT column and purified as follows. An equal volume of 2× oligo dT wash buffer (200 mM Tris, pH 8, 2 M NaCl, 0.1% Tween-20) was added to the translation mix. The mix (up to 1 nmol RNA per translation mix) was then transferred to 100 mg (1 mL) pre-washed oligo dT cellulose (Amersham Pharmacia, catalog number 27-5543-03). The oligo dT/translation mixtures were then rocked at 4° C. for one hour and 15 minutes. Mixtures were spun at 2000 rpm for two minutes at 4° C., supernatants were removed and 400 ³⁵S wash buffer (100 mM Tris pH 8, 1 M NaCl, 0.05% Tween-20) was added. Mixtures were then loaded onto a drip/spin column (BioRad, catalog number 732-6204) and then spun in a microfuge at 1000 rpm for ten seconds. The column was washed seven times with oligo dT wash buffer, spinning after each wash as above. Elution was performed as follows: Elution (E)1=60 μL dH20, spin at 1000 rpm for 10 seconds; E2=500 μL dH20, spin at 3000 rpm for 20 seconds; E3=300 μL dH20, spin at 3000 rpm for 20 seconds. Fusion protein production was estimated using 5 μL of input, last wash, E1, E2, and E3 in a scintillation counter. The following equation was used to determine the concentration of the fusion protein: Pmol fusion=(E_{count} X Eᵥₒₗᵤₘₑ X 1030 X # of 200 uL lysate used)/Input_{count} X Inputᵥₒₗᵤₘₑ.

Reverse transcription of the purified product was also performed using SuperScript Transcriptase from Invitrogen (catalog number 18064-014) to confirm the presence of the appropriate purified fusion protein in each sample. 830 μL of E2 and E3 from the oligo dT column was combined with 1 μL of RT primer (1 mM; HuFn3′ flagstop), 249 μL of 5× first strand buffer, 112.5 μL dH20, 12.5 μL 0.1 M DTT, 25 μL 25 mM dNTPs, and 25 μL of Superscript II in a final volume of 1255 μL. The reaction was incubated for 75 minutes at 42° C.

Translated fusion proteins were further purified using the FLAG tag incorporated into the original cDNA construct (FIG. 8A). 500 μL of M2 agarose (Sigma, catalog number A1205) was transferred to a 1.5 mL tube and spun in a microfuge for 20 seconds at 1000 rpm. The agarose was then washed once with 1 mL Flag binding buffer (50 mM HEPES pH 7.4, 150 mM NaCl, 0.02% Triton X-100) and the follow mixture was added to the agarose: 1250 μL of reverse transcription sample (note-volume is without chemical modification), 312.5 μL of 5× Flag binding buffer (250 mM HEPES pH 7.4, 750 mM NaCl, 0.1% Triton) and rocked at 4° C. overnight. The mixtures were spun in a microfuge at 1000 rpm for 20 seconds and supernatants were transferred to a fresh tube. 0.5 mL Flag binding buffer was added to the tube containing M2 agarose and loaded onto a spin column, spun in a microfuge at 1000 rpm for 10 seconds and washed five times with 0.5 mL Flag binding buffer. Samples were eluted with 500 μL of 100 μg/mL FLAG peptide in Selection buffer (Flag binding buffer+1 mg/ml BSA, 100 μg/mL ssDNA). Samples were eluted again with 300 μL of 100 μg/mL FLAG peptide in Selection buffer as above. The percent recovery was then determined by scintillation counting of the loaded sample, the last wash and elutions 1 and 2.

The final FLAG-purified product was then ready to use in the binding reaction described below. Table 4 shows the efficiency and yield of each step in the fusion protein production for all eight samples.

TABLE 4
Fusion protein production: yields and efficiency.

| | | | Total | Translation | | Oligo dT | Post- | FLAG | |
|---|---|---|---|---|---|---|---|---|---|
| | | RNA conc. | RNA | Mix | Post-Oligo | Efficiency | FLAG | Efficiency | Binding to M280 |
| | Name | (pmol/μL) | (nmol) | (pmol) | dT (pmol) | (%) | (pmol) | (%) | beads (%) |
| #1 | HSA I | 6.2 | 6.2 | 600 | 16.3 | 2.7 | 3.5 | 21.3 | 0.14 |
| #2 | HSA IB | 14.1 | 14.1 | 600 | 27.5 | 4.6 | 7.0 | 25.3 | 0.28 |
| #3 | HSA II | 8.0 | 8.0 | 600 | 25.0 | 4.2 | 6.9 | 27.8 | 0.18 |
| #4 | HSA IIA | 12.7 | 12.7 | 600 | 42.0 | 7.0 | 8.4 | 20.0 | 0.23 |
| #5 | HSA IIB | 12.2 | 12.2 | 600 | 23.3 | 3.9 | 7.1 | 30.5 | 0.15 |
| #6 | HSA III | 7.0 | 7.0 | 600 | 11.2 | 1.9 | 2.5 | 22.5 | 0.12 |
| #7 | HSA IIIA | 10.5 | 10.5 | 600 | 13.4 | 2.2 | 3.7 | 27.3 | 0.12 |
| #8 | HSA IIIB | 9.9 | 9.9 | 600 | 18.1 | 3.0 | 7.4 | 40.9 | 0.06 |

Fusion proteins purified by the methods described above were used in a simulated binding reaction to determine if the proteins would have a non-specific affinity for beads used in RNA-protein fusion target binding selection protocols. After FLAG purification, 2 pmol of each of the eight ³⁵S labeled HSA scaffold based fusion proteins was added to Selection Buffer (50 mM HEPES pH 7.4, 150 mM NaCl, 0.02% triton X-100, 1 mg/mL) and incubated with 200 μL of washed M280 streptavidin coated magnetic beads (Dynabeads, Dynal, Oslo, Norway) and incubated at 30° C. for one hour. The beads were then washed five times for one minute with Selection Buffer (50 mM HEPES pH 7.4, 150 mM NaCl, 0.02% Triton X-100) plus 2 mM biotin. After washing, the M280 beads were diluted in 100 μL Selection Buffer containing 2 mM biotin, and 20 μL of each sample was counted in a scintillation counter to measure the amount of ³⁵S labeled protein that remained on the beads. As is shown in Table 4, less than 1.0% of each of the serum albumin scaffold-based fusion proteins remained bound to the beads indicating minimal, if any, non-specific affinity of the serum albumin scaffold-based fusion proteins for the streptavidin coated beads.

PCR reactions were also performed on the flow-through of the fusion protein/streptavidin bead mixture to analyze the efficiency of the reverse transcription reaction. As a positive control, a binding reaction using an Hf02 library and TNF-α and VEGF as targets was performed. Fusion proteins from the Hf02 library were incubated with biotinylated TNF-α or VEGF for one hour at 30° C. The target-fusion complexes were then captured on streptavidin-coated magnetic beads and flow-through was collected for PCR reactions. The standard PCR reaction for each sample included 18.3 μL dH20, 2.5 μL Herculase Buffer (Stratagene), 0.2 μL dNTP mix containing 25 mM of each NTP, 0.5 μL of a 5′ primer (10 μM) and 0.5 μL of a 3′ primer (10 μM), 2.5 μL of the flow through from the binding reaction diluted 1:25 with water and 0.5 μL of Hot Start Herculase (Stratagene). The temperature cycling program for the PCR reaction was as follows: 95° C. for three minutes; followed by 15, 20, or 25 cycles of 95° C. for 30 seconds, 55° C. for 30 seconds, and 72° C. for three minutes; followed by a five minute extension at 72° C.; and samples were then held at 4° C. Products were separated on a 2% E-gel.

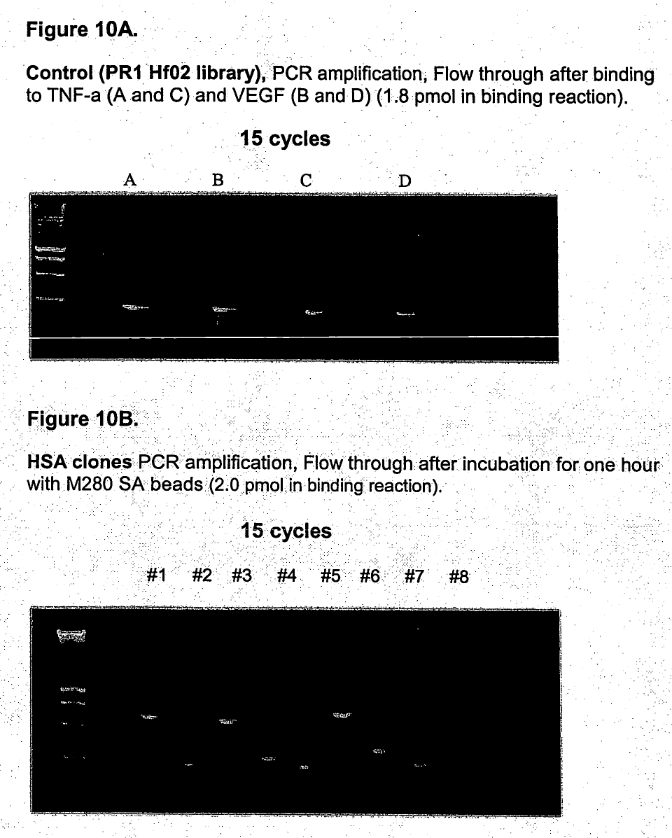

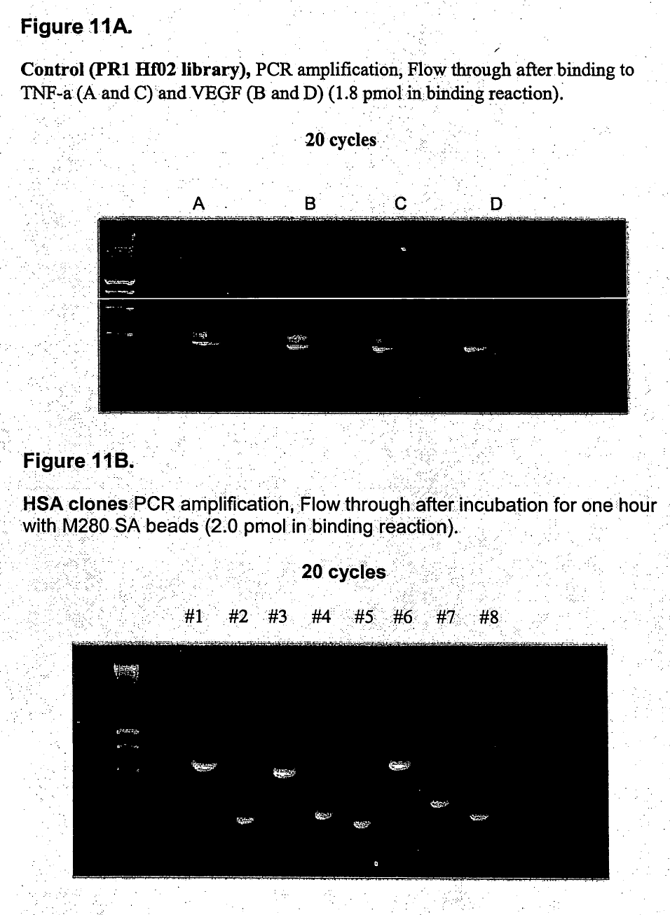

FIG. 10 shows the gels with the PCR products for the flow-through from both the control (FIG. 10A) and the serum albumin scaffold-based fusion proteins (FIG. 10B) after 15 cycles of PCR amplification. FIG. 11 shows the results after 20 cycles of PCR amplification. The results demonstrated that the efficiency of the reverse transcription reactions for the eight tested constructs was similar to the control reaction using a well-established Hf02 library (i.e., the same number of PCR cycles were required for amplification from the flow-through). These results demonstrated the presence of the serum albumin scaffold-based fusion proteins in the flow through after incubation with the streptavidin coated beads. Similar to the results obtained by scintillation counting of the protein remaining on the beads, these results suggest that very little, if any, of the fusion proteins had a non-specific binding affinity for the coated beads.

1. A library of serum albumin scaffold-based candidate binding proteins, said candidate binding proteins having sequences derived from the human serum albumin (HSA) protein sequence of FIG. 1 (SEQ ID NO: 1), wherein each of said candidate binding proteins has one or more mutated amino acids within at least one of the serum albumin domains or sub-domains.

2. The library of claim 1, wherein at least one candidate binding protein has a mutation in sub-domain IB of HSA.

3. The library of claim 1, wherein at least one candidate binding protein has a mutation in sub-domain IIB of HSA.

4. The library of claim 1, wherein at least one candidate binding protein has a mutation in sub-domain IIIB of HSA.

5. The library of claim 1, wherein at least one candidate binding protein has a mutation in sub-domain IA of HSA.

6. The library of claim 1, wherein at least one candidate binding protein has a mutation in sub-domain IIA of HSA.

7. The library of claim 1, wherein at least one candidate binding protein has a mutation in sub-domain IIIA of HSA.

8. The library of claim 1, wherein at least one candidate binding protein has a mutation in domain I of HSA.

9. The library of claim 1, wherein at least one candidate binding protein has a mutation in domain II of HSA.

10. The library of claim 1, wherein at least one candidate binding protein has a mutation in domain III of HSA.

11. The library of claim 1, wherein at least one candidate binding protein has a mutation in full-length HSA.

12. The library of claim 1, wherein at least one candidate binding protein has mutations in more than one HSA sub-domains.

13. The library of claim 1, wherein at least one candidate binding protein has mutations in two or more amino acids.

14. The library of claim 13, wherein at least one candidate binding protein has mutations in six or more amino acids.

15. The library of claim 14, wherein said at least one candidate binding protein has mutations in no more than 50% of the total amino acids.

16. The library of claim 1, wherein at least one candidate binding protein has a mutated HSA sub-domain fused to the remaining wild-type HSA protein.

17. The library of claim 1, wherein at least one candidate binding protein has a mutated HSA sub-domain fused to at least one additional HSA sub-domain.

18. The library of claim 1, wherein at least one candidate binding protein has a binding affinity for a compound that is at least ten-fold higher than the binding affinity of wild-type HSA for said compound.

19. The library of claim 18, wherein said compound is tumor necrosis factor α (TNF-α).

20. The library of claim 1, wherein at least one candidate binding protein is part of a fusion protein.

21. The library of claim 20, wherein said fusion protein further comprises a complement protein.

22. The library of claim 20, wherein said fusion protein further comprises a toxin protein.

23. The library of claim 1, wherein the candidate binding proteins of said library are immobilized on a solid support.

24. A library of serum albumin scaffold-based proteins, wherein each of said candidate binding proteins has a sequence derived from the human serum albumin (HSA) protein sequence of FIG. 1 (SEQ ID NO: 1) by randomizing a sub-domain or a domain of HSA.

25. The library of claim 24, wherein at least one candidate binding protein comprises more than one randomized HSA sub-domains.

26. A library of nucleic acid-protein fusion molecules, the proteins of said molecules being derived from the human serum albumin sequence of FIG. 1 (SEQ ID NO: 1), wherein each of the proteins has one or more mutated amino acids within at least one of the serum albumin domains or sub-domains, the proteins being attached to the nucleic acids of the fusion molecules by means of a covalent bond.

27. The library claim 26, wherein said nucleic acid comprises DNA.

28. The library of claim 26, wherein said nucleic acid comprises RNA.

29. The library of claim 28, wherein said RNA is an mRNA.

30. The library of claim 26, wherein said nucleic acid of the fusion molecule encodes the protein to which it is bound.

31. The library of claim 26, wherein the nucleic acid-protein fusion molecules of said library are immobilized on a solid support.

32. A library of nucleic acid-protein fusion molecules, wherein the proteins of said molecules are derived from the human serum albumin (HSA) sequence of FIG. 1 (SEQ ID NO: 1) by randomizing a sub-domain or a domain of HSA, and wherein the proteins are attached to the nucleic acids of the fusion molecules by means of a covalent bond.

33. The library of claim 32, wherein the protein of at least one of said nucleic acid-protein fusion molecules comprises more than one randomized HSA sub-domains.

34. A method for obtaining a protein which binds to a compound, said method comprising:
(a) contacting a compound with a library of serum albumin scaffold-based candidate binding proteins under conditions that allow binding to form a compound-protein complex, said proteins being derived from the human wild-type serum albumin protein sequence of FIG. 1 (SEQ ID NO: 1), wherein each of said candidate binding proteins has one or more mutated amino acids within at least one of the serum albumin domains or sub-domains; and
(b) obtaining, from said complex, a protein that binds to said compound.

35. The method of claim 34, said method further comprising mutating at least one sub-domain of the protein obtained in step (b) and repeating steps (a) and (b) using the further mutated protein.

36. The method of claim 34, wherein said compound is a protein.

37. The method of claim 36, wherein said protein is TNF-α.

38. The method of claim 34, wherein each of said candidate binding proteins is covalently bound to a nucleic acid.

39. The method of claim 38, wherein said nucleic acid is DNA.

40. The method of claim 38, wherein said nucleic acid is RNA.

41. The method of claim 38, wherein said nucleic acid encodes the protein to which it is bound.

42. The method of claim 34, wherein the candidate binding proteins of said library are immobilized on a solid support.

43. A method for obtaining a protein which binds to a compound, said method comprising:
(a) contacting a compound with a library of serum albumin scaffold-based candidate binding proteins under conditions that allow binding to form a compound-protein complex, said candidate binding proteins being derived from the human wild-type serum albumin protein sequence of FIG. 1 (SEQ ID NO: 1) by randomizing a sub-domain or domain of HSA; and
(b) obtaining, from said complex, a protein that binds to said compound.

44. The method of claim 43, wherein at least one of the candidate binding proteins of said library comprises more than one randomized HSA sub-domains.


SEQUENCE LISTING (1 sequence)

SEQ ID NO: 1 - protein, 583 aa, Human serum albumin
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSWLLLRLAKTYKTTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSWLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL